(19) United States
(12) Patent Application Publication
(10) Pub. No.: US 2005/0271697 A1
(43) Pub. Date: Dec. 8, 2005

(54) Local delivery of growth factors for stem cell transplantation

(75) Inventor: Frank Litvack, Los Angeles, CA (US)

Correspondence Address: CINDY A. LYNCH;CONOR MEDSYSTEMS, INC., 1003 HAMILTON COURT, MENLO PARK, CA, 94025, US

(73) Assignee: Conor Medsystems, Inc., Menlo Park, CA (US)

(21) Appl. No.: 11/148,002

(22) Filed: Jun. 7, 2005

Related U.S. Application Data
(60) Provisional application No. 60/578,122, filed on Jun. 7, 2004.

(51) Int. Cl. A61K 38/18; A61F 2/00

(52) U.S. Cl. 424/423; 514/2

(57) ABSTRACT

A method and apparatus for local delivery of growth factors which enhances stem cell regeneration of the heart is disclosed. In one example, a stent containing growth factor within openings in the stent delivers the growth factor into a coronary artery to improve effectiveness of the stem cell transplantation therapy. The stent may also be used to transplant stem cells and deliver other bioactive factors.

### CROSS-RELATION TO RELATED APPLICATION

This application claims priority based on Provisional Application Ser. No. 60/578,122, entitled “Local Delivery Of Growth Factors For Stem Cell Transplantation” by Frank Litvack, filed on Jun. 7, 2004.

### FIELD OF THE INVENTION

The present invention relates to devices and methods for regenerating cardiac tissue. More particularly, the present invention relates to the local delivery of growth factors within coronary arteries to increase the effectiveness of stem cell transplantation or regeneration in the treatment of myocardial infarction and congestive heart failure.

### BACKGROUND OF THE INVENTION

The reduction or cessation of blood flow to a vascular bed accounts for a variety of clinical events that require immediate intervention and restoration of adequate perfusion to the jeopardized organ or tissue. Impaired perfusion of cardiac tissue (ischemia) due to acute myocardial infarction (MI) results in a loss of the heart's ability to function properly as the tissue becomes oxygen and energy deprived. Permanent injury is directly related to the duration of the oxygen deficit the myocardium experiences. Ischemia occurs when blood flow to an area of cells is insufficient to support normal metabolic activity. Surgical and percutaneous revascularization techniques following acute MI are highly effective in treating ischemic myocardial tissue. In the case of an acute MI, the main blood flow is stopped by the blockage of a coronary artery and the tissue is perfused only through collateral arteries. If the ischemic condition persists for an extended period, the damage to cells within the ischemic zone progresses to irreversible injury and cellular necrosis. Reperfusion may be achieved by a blood flow recanalization therapy, generally including one of coronary angioplasty, administration of a thrombolytic drug, coronary artery bypass surgery, or the like. Timely reperfusion of ischemic myocardium limits infarct size and early reperfusion with angioplasty or thrombolytic therapy provides benefits of reduced myocardial damage, improved ventricular function, and reduced mortality in patients with acute MI. Myocardial salvage can however be compromised by such complications as coronary reocclusion and severe residual coronary stenosis.

When reperfusion is not successful acute MI leads to congestive heart failure. Congestive heart failure is a condition in which the heart cannot pump out all of the blood that enters it, which leads to an accumulation of blood in the vessels and fluid in the body tissues. Congestive heart failure has a high morbidity rate. Current therapy for congestive heart failure includes pharmacotherapy including angiotensis-converting enzyme inhibitors and beta-blockers, or heart transplantation.

Various studies have demonstrated the potential to regenerate myocardium and improve perfusion of heart tissue by stem-cell transplantation therapy. Human embryonic stem (hES) cells, derived from the inner cell mass of blastocyst stage embryos, have the capacity to replicate indefinitely. This makes it feasible to culture them on a large scale, a prerequisite for cell transplantation therapy.

However, hES cells are mainly derived from IVF surplus embryos meaning that they are of allogenic origin, which may result in rejection by the recipient's immune system. Also, the isolation and use of embryonic stem (ES) cells still are a topic of intense ethical debate. Moreover, ES cells are by definition tumorgenic when undifferentiated.

In various tissues of the adult body, multipotent stem cells exist that can give rise to new stem cells (self-renewal) and differentiated cells necessary for maintenance or for the restoration of damaged tissue. Somatic stem cells were previously believed to differentiate only into cells characteristic of the tissue wherein they reside. However, recent experiments provide evidence that certain somatic stem cells, including heart, bone marrow, fat, liver, skin, brain, skeletal muscle, pancreas, and peripheral blood, can differentiate into cells other than those of their tissue of origin.

Adult stem cells are present only in small quantities in the body and are difficult to isolate and purify. There is also evidence that adult stem cells do not function as efficiently or proliferate as quickly as do younger cells. They potentially have DNA abnormalities as a result of years of exposure to environmental toxins and errors in DNA replication. Also of concern is the knowledge that most available adult stem cells are multipotent rather than pluripotent, limiting their ability to develop into specialized cells, such as cardiomyocytes. In fact, multipotent stem cells may develop into noncardiac tissues that become incorporated into the regenerating myocardium with the potential for unknown deleterious cardiac effects, such as conduction delays or dysrhythmias.

Analysis of post-transplant organs of sex-mismatched heart transplantations suggests that there is a circulating pool of stem cells that may regenerate cardiac muscle. Cardiomyocytes and vascular cells derived from the male recipients of female donor hearts were observed. Y-chromosome containing primitive cells, expressing Sca-1m MDR-1 and c-Kit were also sporadically observed. Quaini F. et al., “Chimerism of the transplanted heart,” N. Engl. J. Med., 346: 5-15, 2002.

A recent study also indicates that active endogenous cardiac stem cells are present in the heart even in advanced stages of failure. Urbanek et al., “Intense myocyte formation from cardiac stem cells in human cardiac hypertrophy,” Proc Natl Acad Sci USA. 100(18):10440-5, 2003.

Many researchers have expressed concerns about stem cell treatments, pointing to the recent discovery that stem cells from bone marrow may fuse with other cells. This fusion could produce unstable cells that might trigger disease, according to some researchers. Bone marrow stem cells may fuse with liver cells to produce hybrid cells that contain abnormal numbers of chromosomes. Bone marrow cells may also proliferate a tumor that may be present in a patient. A means of local delivery of growth factors to the coronary arteries to stimulate transplanted, circulating, or endogenous cardiac stem cells is needed.

### BRIEF DESCRIPTION OF THE INVENTION

A solution is provided for local delivery of growth factors that enhance stem cell regeneration of the heart. Stem cells and other bioactive factors may also be locally delivered in accordance with the disclosure. In one embodiment, a stent containing growth factor within openings in the stent delivers the growth factor into a coronary artery to improve effectiveness of the stem cell transplantation therapy.

### BRIEF DESCRIPTION OF THE DRAWINGS

The accompanying drawings, which are incorporated into and constitute a part of this specification, illustrate one or more embodiments of the present invention and, together with the detailed description, serve to explain the principles and implementations of the invention.

FIG. 1 is a cross-sectional perspective view of a portion of an expandable medical device implanted in the lumen of an artery with a therapeutic agent arranged for delivery to the lumen of the artery.

FIG. 2 is a perspective view of an expandable medical device showing a plurality of openings.

FIG. 3 is an expanded side view of a portion of the expandable medical device of FIG. 2.

FIG. 4 is an enlarged cross-section of an opening illustrating a therapeutic agent for directional delivery to a lumen of a blood vessel.

FIG. 5 is an enlarged cross-section of an opening illustrating a first therapeutic agent provided for delivery to a lumen of the blood vessel and a second therapeutic agent provided for delivery to a wall of the blood vessel.

FIG. 6 is an enlarged cross-section of an opening illustrating first and second therapeutic agents for delivery to a lumen of the blood vessel.

### DETAILED DESCRIPTION

A method and apparatus for the local delivery of growth factors improves stem-cell transplantation therapy for treatment of ischemic myocardium and congestive heart failure. A local delivery device is used for delivery of the stem cells into a coronary artery which feeds the location of the transplanted stem cells. In one example, a stent containing growth factor within openings in the stent delivers the growth factor into a coronary artery to improve effectiveness of the stem cell transplantation therapy.

First, the following terms, as used herein, shall have the following meanings:

The terms “drug” and “therapeutic agent” are used interchangeably to refer to any therapeutically active substance that is delivered to a bodily conduit of a living being to produce a desired, usually beneficial, effect.

The term “matrix” or “biocompatible matrix” are used interchangeably to refer to a medium or material that, upon implantation in a subject, does not elicit a detrimental response sufficient to result in the rejection of the matrix. The matrix typically does not provide any therapeutic responses itself, though the matrix may contain or surround a therapeutic agent, and/or modulate the release of the therapeutic agent into the body. A matrix is also a medium that may simply provide support, structural integrity or structural barriers. The matrix may be polymeric, non-polymeric, hydrophobic, hydrophilic, lipophilic, amphiphilic, and the like. Non-polymeric matrices include sugars, starches, carbohydrates, and the like. The matrix may be bioresorbable or non-bioresorbable.

The term “bioresorbable” refers to a matrix, as defined herein, that can be broken down by either chemical or physical process, upon interaction with a physiological environment. The matrix can erode or dissolve. A bioresorbable matrix serves a temporary function in the body, such as drug delivery, and is then degraded or broken into components that are metabolizable or excretable, over a period of time from minutes to years, preferably less than one year, while maintaining any requisite structural integrity in that same time period.

The term “openings” includes both through openings and recesses.

The term “pharmaceutically acceptable” refers to the characteristic of being non-toxic to a host or patient and suitable for maintaining the stability of a beneficial agent and allowing the delivery of the beneficial agent to target cells or tissue.

The term “polymer” refers to molecules formed from the chemical union of two or more repeating units, called monomers. Accordingly, included within the term “polymer” may be, for example, dimers, trimers and oligomers. The polymer may be synthetic, naturally-occurring or semisynthetic. In preferred form, the term “polymer” refers to molecules which typically have a M_{W} greater than about 3000 and preferably greater than about 10,000 and a M_{W} that is less than about 10 million, preferably less than about a million and more preferably less than about 200,000. Examples of polymers include but are not limited to, poly-α-hydroxy acid esters such as, polylactic acid (PLLA or DLPLA), polyglycolic acid, polylactic-co-glycolic acid (PLGA), polylactic acid-co-caprolactone; poly (block-ethylene oxide-block-lactide-co-glycolide) polymers (PEO-block-PLGA and PEO-block-PLGA-block-PEO); polyethylene glycol and polyethylene oxide, poly (block-ethylene oxide-block-propylene oxide-block-ethylene oxide); polyvinyl pyrrolidone; polyorthoesters; polysaccharides and polysaccharide derivatives such as polyhyaluronic acid, poly (glucose), polyalginic acid, chitin, chitosan, chitosan derivatives, cellulose, methyl cellulose, hydroxyethylcellulose, hydroxypropylcellulose, carboxymethylcellulose, cyclodextrins and substituted cyclodextrins, such as beta-cyclo dextrin sulfo butyl ethers; polypeptides, and proteins such as polylysine, polyglutamic acid, albumin; polyanhydrides; polyhydroxy alkonoates such as polyhydroxy valerate, polyhydroxy butyrate, and the like.

The term “primarily” with respect to directional delivery, refers to an amount greater than about 50% of the total amount of beneficial agent provided to a blood vessel.

The term “restenosis” refers to the renarrowing of an artery following an angioplasty procedure which may include stenosis following stent implantation.

The term “stem cells” refers to cells with the capacity for unlimited or prolonged self-renewal that can produce at least one type of highly differentiated descendent. Stem cells include for example fetal cardiomyocytes, skeletal myoblasts, endothelial progenitor cells, embryonic stem cells, and adult mesencyhmal cells.

Apparatus for Locally Delivering Growth Factors

Examples of expandable medical devices or stents for the delivery of therapeutic agents are described in U.S. Patent Publication No. 2003/0082680, published Jun. 27, 2002, which is incorporated herein by reference in its entirety.

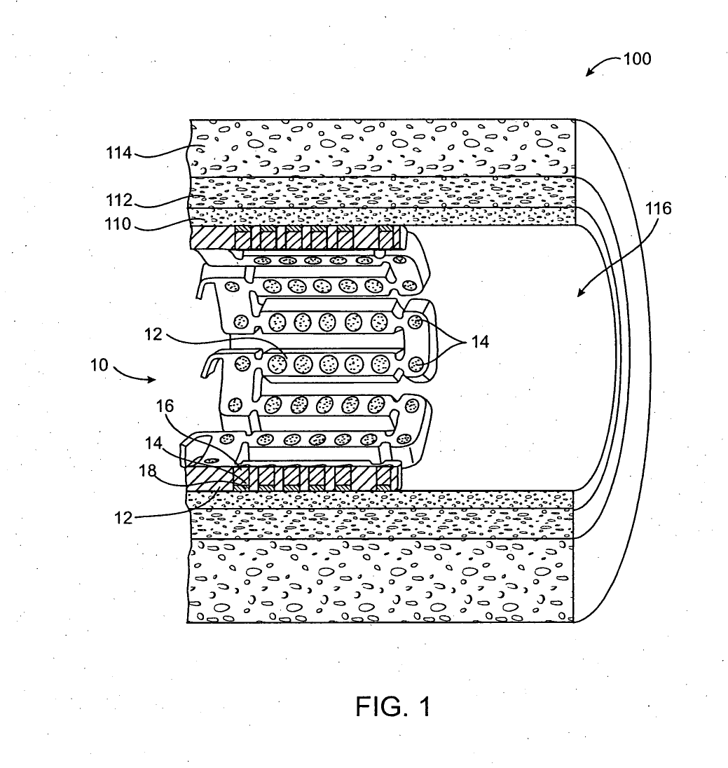

FIG. 1 illustrates an expandable medical device 10 in the form of a stent implanted in a lumen 116 of an artery 100. A wall of the artery 100 includes three distinct tissue layers, the intima 110, the media 112, and the adventitia 114. When the expandable medical device 10 is implanted in a coronary artery directly at, near, or upstream of a site of transplanted or endougenous stem cells, it can be used to deliver growth factors 16 to the stem cells to increase and sustain stem cell proliferation. Growth factor 16 can be delivered by a stent 10 containing drug in openings 14 in the stent. The delivery of the growth factor locally to the heart improves the viability of the transplanted stem cells.

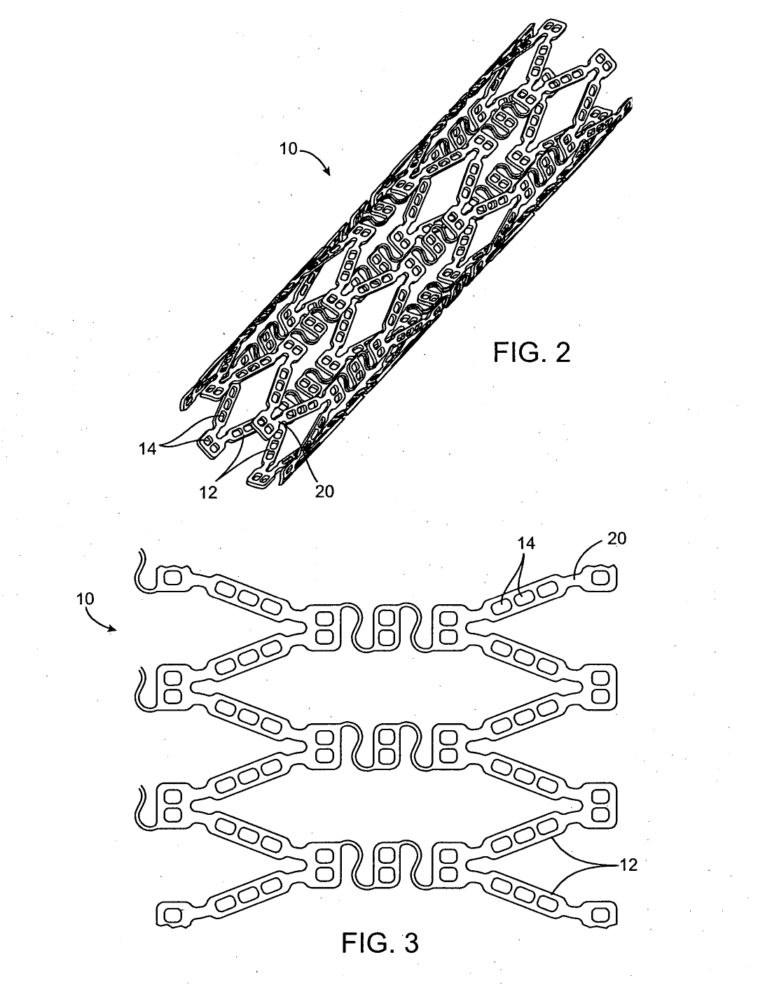

One example of an expandable medical device 10, as shown in FIGS. 1-3, includes large, non-deforming struts 12, which can contain openings 14 without compromising the mechanical properties of the struts, or the device as a whole. The non-deforming struts 12 may be achieved by the use of ductile hinges 20 which are described in detail in U.S. Pat. No. 6,241,762, which is incorporated herein by reference in its entirety. The openings 14 serve as large, protected reservoirs for delivering various beneficial agents to the device implantation site.

The relatively large, protected openings 14, as described above, make the expandable medical device of the present invention particularly suitable for delivering large amounts of therapeutic agents, larger molecules or genetic or cellular agents, and for directional delivery of agents. The large non-deforming openings 14 in the expandable device 10 form protected areas or receptors to facilitate the loading of such an agent, and to protect the agent from abrasion, extrusion, or other degradation during delivery and implantation.

The drug can also be delivered by a drug coated stent, an implant, microspheres, a catheter, coils, or other local delivery means. For example, microspheres, coils, liposomes, or other small drug carriers can be delivered locally at or near the site of a previous occlusion with a catheter or drug delivery stent. These small drug carriers are released and pass downstream into the myocardium where they may implant themselves, delivering the drug directly to myocardial tissue and the transplanted stem cells.

The volume of therapeutic agent that can be delivered using openings 14 is about 3 to 10 times greater than the volume of a 5 micron coating covering a stent with the same stent/vessel wall coverage ratio. The relatively large, protected openings make this stent particularly suitable for delivering large amounts of therapeutic agents, such as the growth factors of the present invention. The larger capacity can be used to deliver multi-drug combinations, each with independent release profiles, for improved efficacy. Also, larger capacity can be used to provide larger quantities of less aggressive drugs and to achieve clinical efficacy without the undesirable side-effects of more potent drugs, such as retarded healing of the endothelial layer.

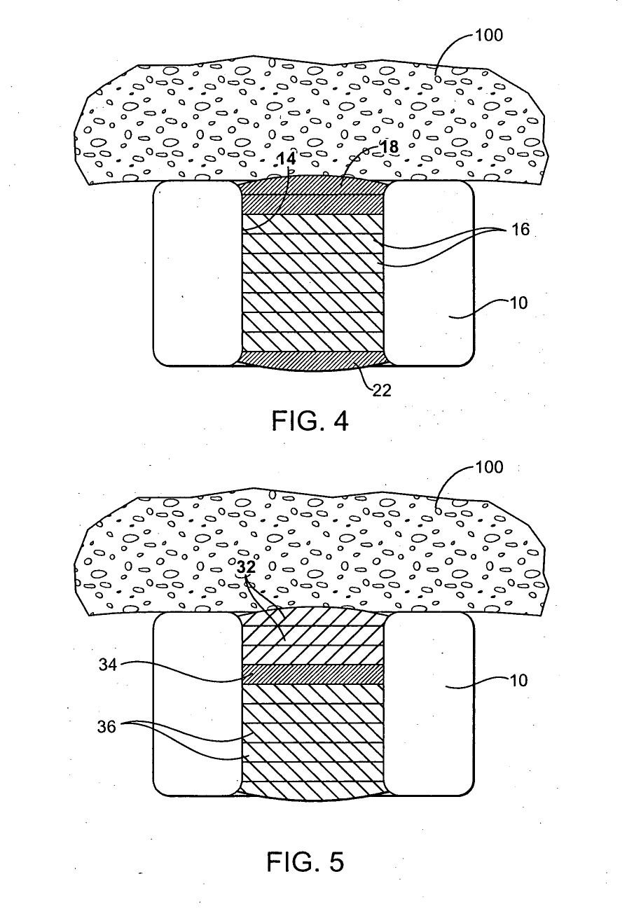

FIG. 4 shows a cross section of a portion of a medical device 10 in which one or more therapeutic agents have been loaded into an opening 14 in multiple layers. Although multiple discrete layers are shown for ease of illustration, the layers may be discrete layers with independent compositions or deposits blended together to form a continuous polymer matrix and agent inlay. For example, multiple deposits of a drug, polymer, solvent composition can be loaded sequentially and then blended together in the openings by the action of the solvent. The agent may be distributed within an inlay uniformly or in a concentration gradient. Examples of some methods of creating such arrangements of layers are described in U.S. Patent Publication No. 2004/0073294, published on Apr. 15, 2004, which is incorporated herein by reference in its entirety. The use of drugs in combination with polymers within the openings 14 allows the medical device 10 to be designed with drug release kinetics tailored to the specific drug delivery profile desired.

According to one example, the total depth of the opening 14 is about 50 to about 140 microns, and the typical deposit thickness would be about 2 to about 50 microns, preferably about 12 microns. Each typical deposit is thus individually about twice as thick as the typical coating applied to surface-coated stents. There can be at least two and preferably about five to twelve such deposit in a typical opening, with a total beneficial agent thickness about 4 to 28 times greater than a typical surface coating. According to one embodiment of the present invention, the openings have an area of at least 5×10⁻⁶ square inches, and preferably at least 10×10⁻⁶ square inches.

In the example of FIG. 4, the mural side of the openings are provided with a barrier 18 of polymer or other material having an erosion rate which is sufficiently slow to allow substantially all of the therapeutic agent 16 to be delivered from the luminal side of the opening prior to complete erosion of the barrier. The barrier 18 prevents loss of the beneficial agent during transport, storage, and during the stent implantation procedure. However, the barrier 18 may be omitted where mural and luminal delivery of the agent is acceptable.

In one example, the barrier 18 and/or the cap 22 may be formed by a material soluble in a different solvent from the therapeutic agent to prevent intermixing of between the barrier, therapeutic agent, and cap. For example, where one or more deposits of therapeutic agent and matrix have been loaded in the openings in a solvent, it may be desirable to select a different polymer and solvent combination for the barrier 18 to prevent the therapeutic agent from mixing into the barrier. Another portion, such as a cap 22 may be formed by a third non-mixing polymer and solvent combination. In addition to the barrier 18 and cap 22, other therapeutic agent deposits, protective deposits, or separating deposits may also be formed of non-mixing polymer/solvent systems in this manner.

A cap 22 can be provided which serves as a seal during filling of the openings. The cap 22 is preferably a rapidly degrading biocompatible material.

Since each deposit of both the barrier 18 and therapeutic agent 16 is created independently, individual chemical compositions and pharmacokinetic properties can be imparted to each deposit. Numerous useful arrangements of such deposits can be formed, some of which will be described below. Each of the deposits may include one or more agents in the same or different proportions from one deposit to the next. Changes in the agent concentration between deposits can be used to achieve a desired delivery profile. For example, a decreasing release of drug for about 24 hours can be achieved. In another example, an initial burst followed by a constant release for about one week can be achieved. Substantially constant release rates over time period from a few hours to months can be achieved. The deposits may be solid, porous, or filled with other drugs or excipients.

In one example, a growth factor is delivered from a stent 10 primarily in a luminal direction with minimal drug being delivered directly from the stent in the direction of the vessel wall. This stent may be placed alone in the occlusion or may be placed in addition to another stent (bare stent or drug delivery stent) placed in connection with an angioplasty procedure.

FIG. 5 is a cross-sectional view of a portion of a stent 10 configured to deliver a second therapeutic agent primarily from a mural side of stent 10, in addition to the growth factor delivered primarily from the luminal side of the stent. The primarily murally delivered agents may include antineoplastics, antiangiogenics, angiogenic factors, immunosuppressants, antirestenotics, anti-thrombotics, such as heparin, antiproliferatives, such as paclitaxel and Rapamycin and derivatives thereof. For example, from about 10 micrograms to about 30 micrograms paclitaxel, useful in the treatment of restinosis, can be delivered morally for at least about 20 days.

In the dual agent example, the drugs can be delivered over different administration periods and at different release rates.

In FIG. 5, an antirestenotic agent 32 is provided at the mural side of the device 10 in one or more deposits and a growth factor 36 is provided at the luminal side of the device in one or more deposits. A separating portion 34 can be provided between the agent deposits. A separating portion 34 can be particularly useful when the administration periods for the two agents are substantially different and delivery of one of the agents will be entirely completed while the other agent continues to be delivered. The separating portion 34 can be any biocompatible material, which is preferably degradable at a rate which is equal to or longer than the longer of the administration periods of the two agents.

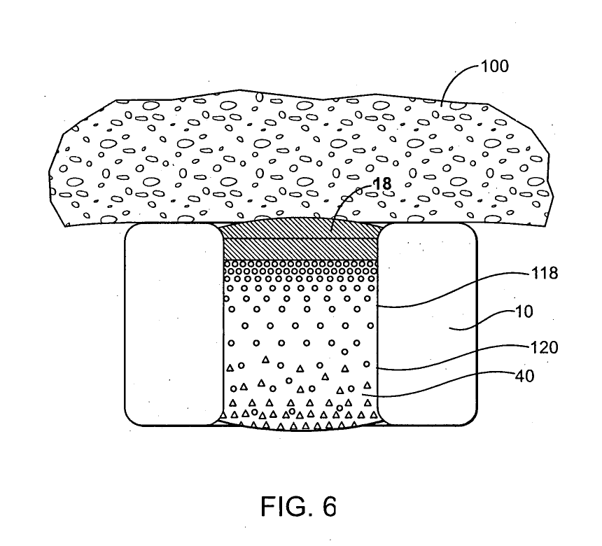

FIG. 6 illustrates an expandable medical device 10 including an inlay 40 formed of a biocompatible matrix with first and second agents provided in the matrix for delivery according to different agent delivery profiles. As shown in FIG. 6, a first drug illustrated by circles 118 is provided in the matrix with a concentration gradient such that the concentration of the drug is highest adjacent the barrier 18 at the mural side of the opening and is lowest at the luminal side of the opening. The second drug illustrated by triangles 120 is relatively concentrated in an area close to the luminal side of the opening. This configuration illustrated in FIG. 6 results in delivery of two different agents with different delivery profiles from the same inlay 40. The two different agents can be agents which treat ischemic injury by different modes of action.

In the embodiments described above, the therapeutic agent can be provided in the expandable medical device in a biocompatible matrix. The matrix can be bioerodible as those described below or can be a permanent part of the device from which the therapeutic agent diffuses. One or more barriers, separating portions, and caps can be used to separate therapeutic agents within the openings or to prevent the therapeutic agents from degradation or delivery prior to implantation of the medical device.

In cases where myocardial infarction is treated by angioplasty, a stent is often delivered to the reopened occlusion site. When the stem cell transplantation therapy is performed or planned, the stent delivered after angioplasty can be a drug delivery stent for delivery of the growth factor. The drug delivery stent for delivery of the growth factor to the transplanted stem cells provides the advantage of increased stem cell proliferation without the difficulties associated with systemic delivery of the growth factor.

Methods for Locally Delivering Growth Factors

The growth factors which are particularly well suited for enhancing the efficacy of stem cell transplantation procedures include, but are not limited to, glia-cell stimulating factor (GSF); colony-stimulating factors (CSF) including macrophage colony-stimulating factor (M-CSF), granulocyte macrophage colony-stimulating factor (GM-CSF), and granulocyte colony-stimulating factor (G-CSF); stem cell growth factor (SCGF) (also called Steel Factor); stromal cell-derived factor (SDF), effective fragments thereof, and combinations thereof; and vascular endothelial growth factor (VEGF). Other growth factors can include hepatocyte growth factor (HGF), Angiopoietin-1, Angiopoietin-2, b-FGF, and FLT-3 ligand, and effective fragment thereof, or DNA coding for such vascularization modulating agents. Disclosure relating to these and other growth factors can be found in Kim, C. H. and Broxmeyer, H. E. Blood, 91:100, 1998; Turner, M. L. and Sweetenham, J. W., Br. J. Haematol. 94:592, 1996; Aiuuti, A. et al., J. Exp. Med. 185:111, 1997; Bleul, C. et al., J. Exp. Med. 184:1101, 1996; Sudo, Y. et al., Blood, 89:3166, 1997; as well as references disclosed therein, each of which is incorporated herein by reference in its entirety. GM-CSF and other hematopoietic factors can potentiate endothelial progenitor cells, or modulate neovascularization. Other growth factors can also include those that induce stem cells to differentiate into myocytes.

Agents for the enhancement of stem cell therapy may also be delivered using a gene therapy-based approach in combination with an expandable medical device. Gene therapy refers to the delivery of exogenous genes to a cell or tissue, thereby causing target cells to express the exogenous gene product. Genes are typically delivered by either mechanical or vector-mediated methods. Mechanical methods include, but are not limited to, direct DNA microinjection, ballistic DNA-particle delivery, liposome-mediated transfection, and receptor-mediated gene transfer. Vector-mediated delivery typically involves recombinant virus genomes, including but not limited to those of retroviruses, adenoviruses, adeno-associated viruses, HVJ (hemagglutinating virus of Japan: Sendai virus), herpesviruses, vaccinia viruses, picornaviruses, alphaviruses, and papovaviruses. Both native and transplanted stem cells may be enhanced using gene therapy to express or overexpress one or more beneficial transgene. The transgene may be for VEGF, basic fibroblast growth factor, heaptocyte growth factor, or other growth factor. The transgene may also be for a cytokine. Cell-based angiogenic gene therapy results in greater angiogenesis and functional improvement than cell transplantation alone. Descriptions of proteins that can be expressed according to the inventive methods may be found in, for example, Human Cytokines: Handbook for Basic and Clinical research, Vol. II (Aggarwal and Gutterman, eds. Blackwell Sciences, Cambridge Mass., 1998); Growth Factors: A Practical Approach (McKay and Leigh, eds., Oxford University Press Inc., New York, 1993) and the Cytokine Handbook (A W Thompson, ed.; Academic Press, San Diego Calif., 1991), each of which is incorporated herein by reference in its entirety.

A growth factor can be released over a sustained administration period which is dependent on the mode of action of the growth factor delivered. For example, a growth factor may be delivered over an administration period of from about 10 hours to 30 days or more. When the growth factor is delivered by a drug delivery stent, the therapeutic dosage is about 5 to about 800 micrograms, for an average stent length of 17 mm, with longer stent being able to deliver additional amounts of growth factor. When the growth factor is delivered locally by a catheter, the dosage can be higher, while the administration period is limited to less than about 24 hours.

Other therapeutic agents for use with the present invention may, for example, take the form of small molecules, peptides, lipoproteins, polypeptides, polynucleotides encoding polypeptides, lipids, protein-drugs, protein conjugate drugs, enzymes, oligonucleotides and their derivatives, ribozymes, other genetic material, cells, antisense oligonucleotides, monoclonal antibodies, platelets, prions, viruses, bacteria, eukaryotic cells such as endothelial cells, stem cells, ACE inhibitors, monocyte/macrophages and vascular smooth muscle cells. Such agents can be used alone or in various combinations with one another. For instance, anti-inflammatories may be used in combination with antiproliferatives to mitigate the reaction of tissue to the antiproliferative. The therapeutic agent may also be a pro-drug, which metabolizes into the desired drug when administered to a host. In addition, therapeutic agents may be pre-formulated as microcapsules, microspheres, microbubbles, liposomes, niosomes, emulsions, dispersions or the like before they are incorporated into the matrix. Therapeutic agents may also be radioactive isotopes or agents activated by some other form of energy such as light or ultrasonic energy, or by other circulating molecules that can be systemically administered.

Exemplary classes of therapeutic agents include antiproliferatives, antithrombins (i.e., thrombolytics), immunosuppressants, antilipid agents, anti-inflammatory agents, antineoplastics including antimetabolites, antiplatelets, angiogenic agents, anti-angiogenic agents, vitamins, antimitotics, metalloproteinase inhibitors, NO donors, nitric oxide release stimulators, anti-sclerosing agents, vasoactive agents, endothelial growth factors, beta blockers, hormones, statins, insulin growth factors, antioxidants, membrane stabilizing agents, calcium antagonists (i.e., calcium channel antagonists), retinoids, anti-macrophage substances, antilymphocytes, cyclooxygenase inhibitors, immunomodulatory agents, angiotensin converting enzyme (ACE) inhibitors, anti-leukocytes, high-density lipoproteins (HDL) and derivatives, cell sensitizers to insulin, prostaglandins and derivatives, anti-TNF compounds, hypertension drugs, protein kinases, antisense oligonucleotides, cardio protectants, petidose inhibitors (increase blycolitic metabolism), endothelin receptor agonists, interleukin-6 antagonists, anti-restenotics, and other miscellaneous compounds.

Antiproliferatives include, for example, sirolimus, paclitaxel, actinomycin D, rapamycin, midostaurin, imatinib mesylate, and cyclosporin. Loaded on stents at a dosage of about 180 micrograms, sirolimus can be effectively used to inhibit smooth muscle cell proliferation—a key contributor to restenosis—after vessel wall balloon/stent injury. Sirolimus derivatives, such as ABT578 and biolimusA9 may also be used. Preclinical studies suggest that ABT578 has potent effects on smooth muscle cell growth and inhibits hyperplasia in a pig model at a dose of 10 micrograms/mm stent. BiolimusA9 is more lipophylic and may be delivered in a PLA polymer.

Antithrombins include, for example, heparin, plasminogen, α₂-antiplasmin, streptokinase, bivalirudin, and tissue plasminogen activator (t-PA).

Immunosuppressants include, for example, cyclosporine, rapamycin and tacrolimus (FK-506), sirolumus, everolimus, etoposide, and mitoxantrone.

Antilipid agents include, for example, HMG CoA reductase inhibitors, nicotinic acid, probucol, and fibric acid derivatives (e.g., clofibrate, gemfibrozil, gemfibrozil, fenofibrate, ciprofibrate, and bezafibrate).

Anti-inflammatory agents include, for example, salicylic acid derivatives (e.g., aspirin, insulin, sodium salicylate, choline magnesium trisalicylate, pimecrolimus, salsalate, dflunisal, salicylsalicylic acid, sulfasalazine, and olsalazine), para-amino phenol derivatives (e.g., acetaminophen), indole and indene acetic acids (e.g., indomethacin, sulindac, and etodolac), heteroaryl acetic acids (e.g., tolmetin, diclofenac, and ketorolac), arylpropionic acids (e.g., ibuprofen, naproxen, flurbiprofen, ketoprofen, fenoprofen, and oxaprozin), anthranilic acids (e.g., mefenamic acid and meclofenamic acid), enolic acids (e.g., piroxicam, tenoxicam, phenylbutazone and oxyphenthatrazone), alkanones (e.g., nabumetone), glucocorticoids (e.g., dexamethaxone, prednisolone, and triamcinolone), pirfenidone, and tranilast.

Antineoplastics include, for example, nitrogen mustards (e.g., mechlorethamine, cyclophosphamide, ifosfamide, melphalan, and chlorambucil), methylnitrosoureas (e.g., streptozocin), 2-chloroethylnitrosoureas (e.g., carmustine, lomustine, semustine, and chlorozotocin), alkanesulfonic acids (e.g., busulfan), ethylenimines and methylmelamines (e.g., triethylenemelamine, thiotepa and altretamine), triazines (e.g., dacarbazine), folic acid analogs (e.g., methotrexate), pyrimidine analogs (5-fluorouracil, 5-fluorodeoxyuridine, 5-fluorodeoxyuridine monophosphate, cytosine arabinoside, 5-azacytidine, and 2′,2′-difluorodeoxycytidine), purine analogs (e.g., mercaptopurine, thioguanine, azathioprine, adenosine, pentostatin, cladribine, and erythrohydroxynonyladenine), antimitotic drugs (e.g., vinblastine, vincristine, vindesine, vinorelbine, paclitaxel, docetaxel, epipodophyllotoxins, dactinomycin, daunorubicin, doxorubicin, idarubicin, epirubicin, mitoxantrone, bleomycins, plicamycin and mitomycin), phenoxodiol, etoposide, and platinum coordination complexes (e.g., cisplatin and carboplatin).

Antiplatelets include, for example, insulin, dipyridamole, tirofiban, eptifibatide, abciximab, and ticlopidine.

Angiogenic agents include, for example, phospholipids, ceramides, cerebrosides, neutral lipids, triglycerides, diglycerides, monoglycerides lecithin, sphingosides, angiotensin fragments, nicotine, pyruvate thiolesters, glycerol-pyruvate esters, dihydoxyacetone-pyruvate esters and monobutyrin.

Anti-angiogenic agents include, for example, endostatin, angiostatin, fumagillin and ovalicin.

Vitamins include, for example, water-soluble vitamins (e.g., thiamin, nicotinic acid, pyridoxine, and ascorbic acid) and fat-soluble vitamins (e.g., retinal, retinoic acid, retinaldehyde, phytonadione, menaqinone, menadione, and alpha tocopherol).

Antimitotics include, for example, vinblastine, vincristine, vindesine, vinorelbine, paclitaxel, docetaxel, epipodophyllotoxins, dactinomycin, daunorubicin, doxorubicin, idarubicin, epirubicin, mitoxantrone, bleomycins, plicamycin and mitomycin.

Metalloproteinase inhibitors include, for example, TIMP-1, TIMP-2, TIMP-3, and SmaPI.

NO donors include, for example, L-arginine, amyl nitrite, glyceryl trinitrate, sodium nitroprusside, molsidomine, diazeniumdiolates, S-nitrosothiols, and mesoionic oxatriazole derivatives.

NO release stimulators include, for example, adenosine.

Anti-sclerosing agents include, for example, collagenases and halofuginone.

Vasoactive agents include, for example, nitric oxide, adenosine, nitroglycerine, sodium nitroprusside, hydralazine, phentolamine, methoxamine, metaraminol, ephedrine, trapadil, dipyridamole, vasoactive intestinal polypeptides (VIP), arginine, and vasopressin.

Endothelial growth factors include, for example, VEGF (Vascular Endothelial Growth Factor) including VEGF-121 and VEG-165, FGF (Fibroblast Growth Factor) including FGF-1 and FGF-2, HGF (Hepatocyte Growth Factor), and Ang1 (Angiopoietin 1). Schumacher et al. injected 0.01 mg/kg body weight of FGF-1 in 20 patients with triple vessel disease close to the internal mammary artery to the left anterior descending artery or one of its diagonal branches. At 12 weeks, the internal mammary artery bypasses were selectively imaged by intra-arterial digital subtraction angiography and quantitatively evaluated. In all the patients, a capillary network sprouting from the proximal part of the coronary artery could be shown to have bypassed the stenosis and rejoined the distal part of the vessel with a resultant 2 to 3-fold increase in the local blood supply. Schumacher et al., “Induction of neoangiogenesis in ischemic myocardium by human growth factors: first clinical results of a new treatment of coronary heart disease,” Circulation, 97:645-50, 1998.

Beta blockers include, for example, propranolol, nadolol, timolol, pindolol, labetalol, metoprolol, atenolol, esmolol, and acebutolol.

Hormones include, for example, progestin, insulin, the estrogens and estradiols (e.g., estradiol, estradiol valerate, estradiol cypionate, ethinyl estradiol, mestranol, quinestrol, estrond, estrone sulfate, and equilin).

Statins include, for example, mevastatin, lovastatin, simvastatin, pravastatin, atorvastatin, and fluvastatin.

Insulin growth factors include, for example, IGF-1 and IGF-2.

Antioxidants include, for example, vitamin A, carotenoids and vitamin E.

Membrane stabilizing agents include, for example, certain beta blockers such as propranolol, acebutolol, labetalol, oxprenolol, pindolol and alprenolol.

Calcium antagonists include, for example, amlodipine, bepridil, diltiazem, felodipine, isradipine, nicardipine, nifedipine, nimodipine and verapamil.

Retinoids include, for example, all-trans-retinol, all-trans-14-hydroxyretroretinol, all-trans-retinaldehyde, all-trans-retinoic acid, all-trans-3,4-didehydroretinoic acid, 9-cis-retinoic acid, 11-cis-retinal, 13-cis-retinal, and 13-cis-retinoic acid.

Anti-macrophage substances include, for example, NO donors.

Anti-leukocytes include, for example, 2p-CdA, IL-1 inhibitors, anti-CD116/CD18 monoclonal antibodies, monoclonal antibodies to VCAM, monoclonal antibodies to ICAM, and zinc protoporphyrin.

Cyclooxygenase inhibitors include, for example, Cox-1 inhibitors and Cox-2 inhibitors (e.g., CELEBREX® and VIOXX®).

Immunomodulatory agents include, for example, immunosuppressants (see above) and immunostimulants (e.g., levamisole, isoprinosine, Interferon alpha, and Interleukin-2).

ACE inhibitors include, for example, benazepril, captopril, enalapril, fosinopril sodium, lisinopril, quinapril, ramipril, and spirapril.

Cell sensitizers to insulin include, for example, glitazones, P par agonists and metformin.

Antisense oligonucleotides include, for example, resten-NG.

Cardio protectants include, for example, VIP, pituitary adenylate cyclase-activating peptide (PACAP), apoA-I milano, amlodipine, nicorandil, cilostaxone, and thienopyridine.

Petidose inhibitors include, for example, omnipatrilat.

Anti-restenotics include, for example, include vincristine, vinblastine, actinomycin, epothilone, paclitaxel, and paclitaxel derivatives (e.g., docetaxel).

Miscellaneous compounds include, for example, Adiponectin.

Methods of Transplantation of Stem Cells

The implantable medical devices with growth factors and the methods of delivery of growth factors of the present invention can be used in conjunction with the transplantation of stem cells to the myocardial tissue.

Skeletal myoblasts can be isolated from muscle tissue and expanded ex vivo to yield a large number of cells for implantation. Both in vitro and in vivo, the myoblasts are able to fuse and form myotubes.

Bone marrow stromal cells or mesenchymal stem cells exist at low frequencies in the bone marrow. However, they readily attach to plastic dishes and proliferate rapidly to yield large number of cells as well. Preliminary long-term studies with bone marrow-derived cells, however, indicate that the new myocytes do not acquire the adult phenotype but resemble neonatal cells, which die with time by apoptosis. Dawn et al., “Cardiac stem cells delivered intravascularly traverse the vessel barrier, regenerate infarcted myocardium, and improve cardiac function,” Proc Natl Acad Sci USA., 102(10):3766-71, 2005. Li et al. demonstrated that myogenic differentiation of CD117⁺ bone marrow stem cells was increased dramatically by TGF-[beta] preprogramming, but were unable to demonstrate that these myogenic differentiated CD117⁺ bone marrow stem cells were really mature cardiomyocytes with normal contractile properties after implantation into infarcted myocardium. Li et al., “Regeneration of infarcted myocardium by intramyocardial implantation of ex vivo transforming growth factor-beta-preprogrammed bone marrow stem cells,” Circulation, 111(19):2438-45, 2005.

Mesenchymal stem cells adopt a fibroblast-like morphology to express myogenic markers and to give rise to cardiomyocyte-like cells.

Smooth muscle cells, which may be obtained from a variety of organs may also be used. Notably, vascular smooth muscle cells appear to be unique in capably modifying the local matrix, inducing significant angiogenesis and improving cardiac function. They may be obtained from pieces of saphenous vein or radial artery.

The stem cells may be transplanted by one or more intramyocardial injections during cardiac surgery, as has been performed in various studies. Stamm et al. injected 1.5×10⁶ autologous AC133+ bone marrow cells (BMCs) into the infarct border zone with good results. Stamm et al., “Autologous bone-marrow stem-cell transplantation for myocardial regeneration,” Lancet, 361; 45-6, 2003. Within the border zone, there is a rim of hibernating myocardium that may be revived with revascularization of angiogenesis. Intramyocardial injection may be through both the endocardial and epicardial routes. This technique may be combined with coronary artery bypass grafting (CABG).

Percutaneous catheter-based myocardial injections provide a less invasive method of transplantation of the stem cells into the myocardial tissue. Percutaneous delivery can be used in conjunction with various visualization and/or mappings systems.

Alternately, the stem cell transplantation can be administered by intracoronary injection or intravenous injection. The intracoronary injection delivers the stem cells directly to the coronary arteries by a catheter and can use high pressure injection to encourage passage of the stem cells through the endothelium. Strauer et al. administered autologous BMCs via a balloon catheter advanced in the infarct-related artery. The cells were injected 7 days after the acute event and, to maximize extraction by the distal vascular bed, injections were performed during balloon inflation that prevented antegrade flow and rapid washout. Strauer et al., “Repair of infracted myocardium by autologous intracoronary mononuclear bone marrow cell transplantation in humans,” Circulation, 106, 1913-61, 2002. The intracoronary route seems to be the most effective and least harmful, because the cells injected are selectively directed to the impaired area and no inconvenience resulting form the production of a myocardial lesion or induction of arrhythmia exists.

Further Stem Cell Transplantation and Regeneration Systems

A further stem cell transplantation system uses a drug delivery stent. For example, a drug delivery stent for transplantation of the stem cells can be the same or a different stent from the growth factor delivery stent. In one example, a stent having openings 14 includes multiple openings containing stem cells and other independent openings containing the growth factor. In another example, a stent having openings 14 includes multiple openings containing stem cells, other independent openings containing biological therapeutic agents, and yet other independent openings containing growth factors for those biological therapeutic agents. In a further embodiment, a stent having openings 14 includes multiple openings containing growth factors and other independent openings containing cytokines.

A number of genes for cytokines including hematopoietic growth factors have been successfully cloned. Such cytokines include transforming growth factor-[beta] (TGF-[beta]), bone morphogenetic proteins, erythropoietin, thrombopoietin, colony stimulating factors such as granulocyte colony-stimulating factor (G-CSF), macrophage colony-stimulating factor (M-CSF), granulocyte macrophage colony-stimulating factor (GM-CSF), interleukins such as IL-1, IL-3, IL-4, IL-5, IL-6, IL-9, IL-11, and IL-12, stem cell factor (SCF), and flk-2/flt3 ligand. Lin et al., Proc. Natl. Acad. Sci. USA 82, 7580-7584, 1985; de Sauvage et al., Nature 369, 533-538, 1994; Nagata et al., EMBO J. 5, 575-581, 1986; Wong et al., Science 235, 1504-1508, 1987; Miyatake et al., EMBO J. 4, 2561-2568, 1985; Clarke et al., Nucleic Acids Res. 14, 7897-7914, 1986; Dorssers et al., Gene 55, 115-124, 1987; Yokota et al., Proc. Natl. Acad. Sci. USA 83, 5894-5898, 1986; Campbell et al., Proc. Natl. Acad. Sci. USA 84, 6629-6633, 1987; Yasukawa et al., EMBO J. 6, 2939-2945, 1987; Yang et al., Blood 74, 1880-1884, 1989; Paul et al., Proc. Natl Acad. Sci. USA 87, 7512-7516, 1990; Wolf et al., J. Immunol, 146, 3074-3081, 1991; Anderson et al., Cell 63, 235-243, 1990; Lyman et al., Cell 75, 1157-1167, 1993.

Stem cell transplantation can also be effected by mobilizing bone marrow resident stem cells to the site of the injured myocardium using cytokines such as granulocyte-colony stimulating factor (G-CSF) and stem cell factor (SCF). G-CSF and SCF cause an increase in the release of stem cells from the bone marrow into the peripheral blood circulation.

The timing of stem cell transplantation has been found to be more effective when the initial inflammation from an acute MI has begun to subside. Thus, stem cell transplantation at about 3 or more days after MI in combination with growth factor delivery timed to deliver the growth factor from about the transplantation time and for a sustained time period following transplantation. For example, if a growth factor delivery stent is delivered during or following an angioplasty procedure immediately after the acute MI, the stent can contain a delayed release formulation which begins to release the growth factor at about the time determined to be appropriate for the stem cell transplantation.

Although the invention has been described with respect to treatment of ischemic myocardial tissue, the invention can also be used for stem cell treatment of non-ischemic cardiomyopathy, peripheral vascular disease, and aging.

While the invention has been described in detail with reference to the preferred embodiments thereof, it will be apparent to one skilled in the art that various changes and modifications can be made and equivalents employed, without departing from the present invention.

1. A method for treating ischemic myocardial tissue, the method comprising:
selecting a delivery site within a blood vessel at or upstream of a stem cell location; and
locally delivering a first therapeutic agent to the selected delivery site over an administration period sufficient to stimulate regeneration of myocardial tissue, wherein the first therapeutic agent is a growth factor.

2. The method of claim 1, wherein the growth factor is glia-cell stimulating factor.

3. The method of claim 1, wherein the growth factor is a colony stimulating factor.

4. The method of claim 1, wherein the growth factor is stromal cell-derived factor.

5. The method of claim 1, wherein the growth factor is stem cell growth factor.

6. The method of claim 1, wherein the growth factor is delivered by a stent.

7. The method of claim 1, further comprising transplanting stem cells into myocardial tissue.

8. The method of claim 6, wherein the growth factor is delivered from openings in the stent.

9. The method of claim 6, wherein the growth factor is coated on the stent.

10. The method of claim 6, further comprising transplanting stem cells into myocardial tissue and implanting the stent locally to deliver the growth factor after transplantation of the stem cells.

11. The method of claim 6, further comprising transplanting stem cells into myocardial tissue and implanting the stent locally to deliver the growth factor before or during transplantation of the stem cells.

12. The method of claim 1, further comprising locally enhancing stem cell proliferation using gene therapy.

13. The method of claim 1, further comprising locally delivering a second therapeutic agent.

14. An implantable medical device for delivering a growth factor locally into an artery, the device comprising:
an implantable medical device configured to be implanted within a coronary artery; and
a first therapeutic agent affixed to the implantable medical device, wherein the first therapeutic agent is a growth factor and wherein the growth factor is released at a therapeutic dosage and over an administration period effective to stimulate regeneration of stem cells.

15. The device of claim 14, wherein the implantable medical device is a stent which is expandable within a coronary artery.

16. The device of claim 14, wherein the growth factor is in a biocompatible matrix.

17. The device of claim 16, wherein the biocompatible matrix is a bioresorbable polymer.

18. The device of claim 16, wherein the biocompatible matrix is non-bioresorbable polymer.

19. The device of claim 14, wherein the growth factor is deposited within openings in the implantable medical device.

20. The device of claim 14, wherein the growth factor is deposited on a surface of the implantable medical device.

21. The device of claim 14, wherein the administration period is about 10 hours to about 30 days.

22. The device of claim 14, wherein the therapeutic dosage is about 5 to about 800 micrograms.

23. The device of claim 14, wherein the growth factor is deposited within openings in the implantable medical device and wherein a barrier layer is provided which substantially prevents delivery of the growth factor murally.

24. The device of claim 14, further comprising stem cells affixed to the implantable medical device.

25. The device of claim 14, further comprising cytokines affixed to the implantable medical device.

26. The device of claim 14, further comprising a second therapeutic agent affixed to the implantable medical device.

27. The device of claim 26, further comprising a second growth factor affixed to the implantable medical device.

28. The device of claim 14, further comprising an antirestenotic composition affixed to the implantable medical device.

29. The device of claim 14, wherein the growth factor is glia-cell stimulating factor.

30. The device of claim 14, wherein the growth factor is a colony stimulating factor.

31. The device of claim 14, wherein the growth factor is stromal cell-derived factor.

32. The device of claim 14, wherein the growth factor is stem cell growth factor.

33. The device of claim 14, wherein the stem cells are endogenous cardiac stem cells.

34. The device of claim 26, wherein the second therapeutic agent is an antirestenotic.

35. The device of claim 26, wherein the second therapeutic agent is an antiproliferative.

36. The device of claim 26, wherein the second therapeutic agent is an immunosuppressant.

37. The device of claim 26, wherein the second therapeutic agent is an antimitotic.

38. The device of claim 14, wherein the stem cell is an endogenous stem cell.

39. The device of claim 14, wherein the stem cell is a transplanted stem cell.
